(19) United States
(12) Patent Application Publication
(10) Pub. No.: US 2025/0114083 A1
(43) Pub. Date: Apr. 10, 2025

(54) CONTROL SYSTEM FOR ULTRASONIC HAND PIECE

(71) Applicant: Alcon Inc., Fribourg (CH)

(72) Inventors: Ahmad GAD, Irvine, CA (US); Mikhail OVCHINNIKOV, Dana Point, CA (US); Satish YALAMANCHILI, Irvine, CA (US); Guangyao JIA, Irvine, CA (US)

(21) Appl. No.: 18/899,960

(22) Filed: Sep. 27, 2024

Related U.S. Application Data
(60) Provisional application No. 63/588,951, filed on Oct. 9, 2023.

(51) Int. Cl. A61B 17/00

(52) U.S. Cl. CPC A61B 17/00; A61B 2017/00106

(57) ABSTRACT

A control system for an ultrasonic hand piece is provided. The control system includes an irrigation pressure sensor configured to measure a pressure of an irrigation fluid, an ultrasonic power source configured to provide power to an ultrasonic transducer, and a controller. The controller is configured to receive a selection of an ultrasonic power modality, generate a control signal based on the selected ultrasonic power modality, provide the control signal to the ultrasonic power source, determine an irrigation flow rate from a measured irrigation pressure provided by the irrigation pressure sensor, calculate a thermal index value based on the irrigation flow rate and the selected ultrasonic power modality, and, in response to the thermal index value reaching a threshold value, adjust the control signal based on the thermal index value. The ultrasonic power modality is one of a two dimensional power modality and a three dimensional power modality.

### CROSS-REFERENCE TO RELATED APPLICATIONS

This application claims the benefit of U.S. Provisional Application Ser. No. 63/588,951 (filed on Oct. 9, 2023), the content of which is incorporated herein by reference in its entirety.

### INTRODUCTION

A typical ultrasonic surgical device/system suitable for ophthalmic procedures includes an ultrasonically driven hand piece, an attached hollow working tip, an irrigating sleeve and an electronic control console. The hand piece assembly is attached to the control console by an electric cable and flexible tubing. Through the electric cable, the console varies the power level transmitted by the hand piece to the attached working tip, and the flexible tubing is used to supply irrigation fluid by an irrigation subsystem, and to aspirate fluid and other particles, such as emulsified tissue, etc., from the eye by an aspiration subsystem.

### SUMMARY

In certain embodiments, a control system for an ultrasonic hand piece is provided. The control system includes an irrigation pressure sensor configured to measure a pressure of an irrigation fluid, an ultrasonic power source configured to provide power to an ultrasonic transducer, and a controller coupled to the irrigation pressure sensor and the ultrasonic power source. The controller is configured to receive a selection of an ultrasonic power modality, generate a control signal based on the selected ultrasonic power modality, provide the control signal to the ultrasonic power source, determine an irrigation flow rate from a measured irrigation pressure provided by the irrigation pressure sensor, calculate a thermal index value based on the irrigation flow rate and the selected ultrasonic power modality, and, in response to the thermal index value reaching a threshold value, adjust the control signal based on the thermal index value. The ultrasonic power modality is one of a two dimensional power modality and a three dimensional power modality.

In certain embodiments, the two dimensional power modality is one of a longitudinal power modality and a torsional power modality, and the three dimensional power modality is a combination of the longitudinal power modality and the torsional power modality.

In certain embodiments, the control system also includes a temperature sensor configured to measure a temperature of the irrigation fluid, and calculating the thermal index value is also based on the measured temperature.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1A illustrates an example of a phacoemulsification system, according to certain embodiments.

FIG. 1B illustrates an example of a subsystem of the phacoemulsification system of FIG. 1A, according to certain embodiments.

FIG. 1C is a block diagram and a perspective view of the hand piece of the phacoemulsification system of FIG. 1A and the subsystem of FIG. 1B, according to certain embodiments.

FIG. 2A is a block diagram of one embodiment of a control system of the phacoemulsification system of FIG. 1A, according to certain embodiments.

FIG. 2B is a block diagram of another embodiment of a control system of the phacoemulsification system of FIG. 1A, according to certain embodiments.

FIGS. 3A-3B are graphs depicting an exemplary operation of thermal management by the control systems of FIGS. 2A-2B, according to certain embodiments.

FIG. 4 illustrates a flow diagram of a technique for controlling power supplied to an ultrasonic hand piece, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

In some examples of ultrasonic surgical devices/systems described above, the operative part of the ultrasonically driven hand piece is a centrally-located, hollow resonating bar or horn directly attached to one or more ultrasonic transducers, such as a set of piezoelectric crystals that form a piezoelectric element assembly. The crystals supply the required ultrasonic vibration needed to drive both the horn and the attached working tip during phacoemulsification and are controlled by the console. The crystal/horn assembly is suspended within the hollow body or shell of the hand piece. The hand piece body terminates in a reduced diameter portion or nosecone at the body's distal end. The nosecone is externally threaded to accept the irrigation sleeve. Likewise, the horn bore is internally threaded at its distal end to receive the external threads of the working tip. The irrigation sleeve also has an internally threaded bore that is screwed onto the external threads of the nosecone. The working tip is adjusted so that the tip projects only a predetermined amount past the open end of the irrigating sleeve.

When used to perform phacoemulsification, the ends of the working tip and irrigating sleeve are inserted into a small incision of predetermined width in the cornea, sclera, or other location in the eye tissue in order to gain access to the anterior chamber of the eye. The working tip is ultrasonically vibrated along its longitudinal axis within the irrigating sleeve by the crystal-driven ultrasonic horn, thereby emulsifying upon contact the selected tissue in situ. The hollow bore of the working tip communicates with the bore in the horn which in turn communicates with the aspiration line from the hand piece to the console. A reduced pressure or vacuum source in the console draws or aspirates the emulsified tissue from the eye through the open end of the working tip, the bore of the working tip, the horn bore, and the aspiration line and into a collection device. The aspiration of emulsified tissue is aided by an irrigation fluid, such as a saline flushing solution, etc., that is injected into the surgical site through the small annular gap between the inside surface of the irrigating sleeve and the outside surface of the working tip.

Unwanted heating can occur at the surgical site when too much power is applied to the hand piece with too low of an irrigation flow rate. Friction between the irrigation sleeve and the tip is the primary source of heat. When the tip rubs against the sleeve, it produces heat. Because different power modalities involve different shapes and/or movements of the tip, the increase in heat at the surgical site may vary due to different thermal behaviors of each power modality. In such cases, thermal management techniques may not account for said different thermal behaviors.

Accordingly, certain embodiments described herein provide systems that prevent overheating at surgical sites by controlling an amount of power provided to a hand piece based on a power modality of the hand piece. The systems account for the power modality of the hand piece when determining a thermal index value at a tip of the hand piece, and automatically reduce the amount of power supplied to the hand piece in response to the thermal index value reaching a threshold value at which burns are likely to occur. By automatically reducing the amount of power supplied when the threshold value is met, the systems reduce the likelihood of burns at the surgical site.

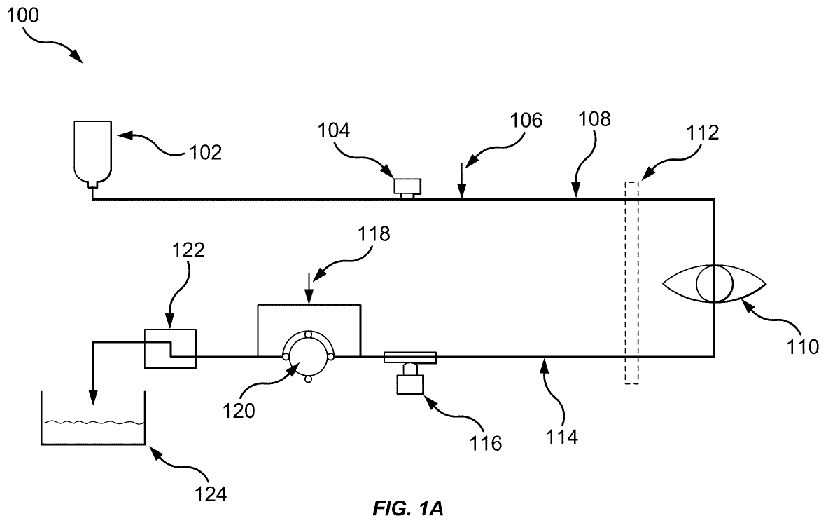

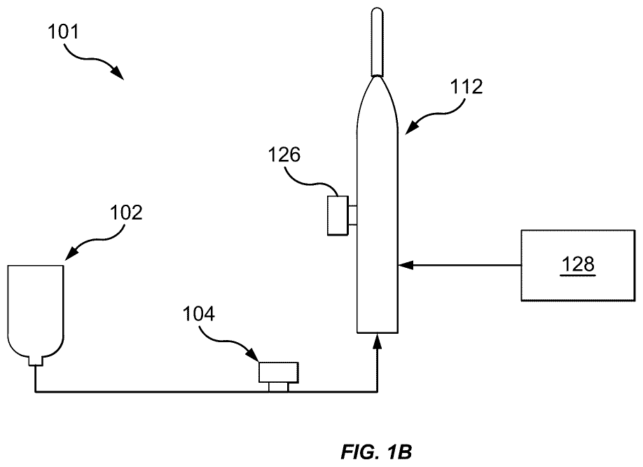

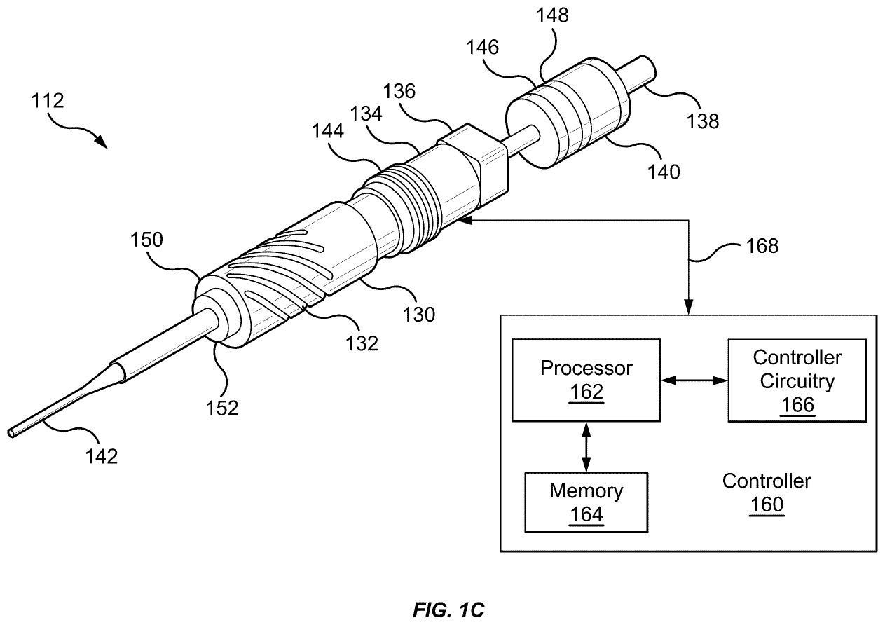

Examples of a phacoemulsification system, a subsystem of the phacoemulsification system, and a hand piece of the phacoemulsification system are described in further detail with reference to FIGS. 1A-1C.

FIG. 1A illustrates an example of a phacoemulsification system 100, according to certain embodiments. The phacoemulsification system 100 may be used to perform ophthalmic procedures on an eye 110. As shown in FIG. 1A, the phacoemulsification system 100 includes components in a fluid path through the eye 110 (e.g., during cataract surgery). In certain embodiments, the components include an irrigation fluid source 102, an irrigation pressure sensor 104, an irrigation valve 106, an irrigation line 108, a hand piece 112, an aspiration line 114, an aspiration pressure sensor 116, a vent valve 118, a pump 120, a reservoir 122, and a drain bag 124. The irrigation line 108 may provide an irrigation fluid, such as a balanced salt solution (BSS), a basic saline solution with a medication, a perfluorocarbon liquid, a viscoelastic substance, or other similar fluid, to the eye 110, and the aspiration line 114 may remove fluid and/or other particles, such as emulsified lens particles, etc., from the eye 110.

When irrigation fluid exits the irrigation fluid source 102, it travels through the irrigation line 108 and into the eye 110 via the hand piece 112. The irrigation pressure sensor 104 may be configured to measure the pressure of the irrigation fluid in the irrigation line 108. The measured pressure of the irrigation fluid may be referred to herein as “measured irrigation pressure” or an “irrigation pressure measurement.” The irrigation valve 106 may also be optionally provided for on/off control of irrigation. The irrigation pressure sensor 104 is implemented by any of a number of commercially available fluid pressure sensors and can be located at any location along the irrigation fluid path between the irrigation fluid source 102 and the eye 110.

The hand piece 112 may be placed in the eye 110 during an ophthalmic procedure, such as a phacoemulsification procedure, etc. The hand piece 112 has a working tip (as seen in FIG. 1C) that may be ultrasonically vibrated in the eye 110 in order to break up a diseased lens, etc. A sleeve located around the working tip provides irrigation fluid from the irrigation line 108. The irrigation fluid passes through the space between the outside of the working tip and the inside of the sleeve. Fluid and/or lens particles are aspirated through the working tip. In this manner, the interior passage of the working tip may be fluidly coupled to the aspiration line 114. The pump 120 draws the aspirated fluid from the eye 110. The aspiration pressure sensor 116 measures the pressure in the aspiration line 114. An optional vent valve 118 can be used to vent the vacuum created by the pump 120. The aspirated fluid passes through the reservoir 122 and into the drain bag 124.

FIG. 1B illustrates an example of a subsystem 101 of the phacoemulsification system 100 (FIG. 1A), according to certain embodiments. The subsystem 101 includes the irrigation fluid source 102, the irrigation pressure sensor 104, and the hand piece 112 as described with reference to FIG. 1B. While the subsystem 101 is shown as including some of the components of the phacoemulsification system 100, the subsystem may include more or less of the components of the phacoemulsification system 100.

In FIG. 1B, a temperature sensor 126 and a power source 128 are coupled to the hand piece 112. The temperature sensor 126 may be configured to measure a temperature of irrigation fluid provided by the hand piece 112. The measured temperature of the irrigation fluid may be referred to herein as a “temperature measurement.” The power source 128 may be an ultrasonic power source or other similar power source configured to provide ultrasonic power to the hand piece 112 to actuate its phacoemulsification tip.

Although the temperature sensor 126 is shown as being separate from the irrigation pressure sensor 104, the temperature sensor 126 and the irrigation pressure sensor 104 may be implemented as one sensor configured to perform the functionalities of both the temperature sensor 126 and the irrigation pressure sensor 104. As an example, the sensor configured to perform the functionalities of both the temperature sensor 126 and the irrigation pressure sensor 104 may be implemented in the hand piece 112, along the irrigation line 108, or at another location within the phacoemulsification system 100.

FIG. 1C is a block diagram of a controller 160 and perspective view of the hand piece 112 of the phacoemulsification system 100 (FIG. 1A) and the subsystem 101 (FIG. 1B), according to certain embodiments. The controller 160 may be configured to control the hand piece 112. The hand piece 112 may be an ultrasonic hand piece which may be used, for example, to perform phacoemulsification. The hand piece 112 is shown with its outer case removed so that components of the hand piece 112 may be seen.

The hand piece 112 includes an ultrasonic horn 130, for example made from a titanium alloy. The horn 130 has a plurality of helical slits 132. At least one ultrasonic transducer, such as a piezoelectric element or crystal, forms an ultrasonic transducer assembly, such as piezoelectric element assembly 134. The piezoelectric elements or crystals may be ring-shaped and may be held by a compression nut 136 against the horn 130. Some hand pieces may include multiple piezoelectric element assemblies 134 that are each physically separated from each other along a longitudinal axis of the hand piece 112, and each may form a separate assembly/packaging. Each piezoelectric element assembly 134 may be separately electrically coupled to the controller 160.

An aspiration shaft or tube 138 extends down the length of the hand piece 112 through the horn 130, the piezoelectric element assembly 134, the nut 136, and the plug 140 at a proximal end of the hand piece 112. The aspiration tube 138 allows material to be aspirated through a hollow working tip 142, which is attached to the horn 130, and through and out the hand piece 112. While the hollow working tip 142 is shown as a straight tip, other tip configurations may also be used, such as a bent tip, etc. The plug 140 seals an outer shell of the hand piece 112 fluid tight, allowing the hand piece 112 to be autoclaved without adversely affecting the piezoelectric element assembly 134. Additional grooves 144 for sealing O-ring gaskets may be provided on the horn 130.

The location of longitudinal and torsional nodal points of the hand piece 112 are indicated on FIG. 1C. The longitudinal and torsional nodal points are the locations experiencing zero velocity of the respective node. The torsional node 146 preferably is located at the proximal longitudinal node 148, so that the torsional node 146 and the longitudinal node 148 are coincident and located on the plug 140. The hand piece 112 also includes a distal longitudinal node 150 located at a reduced diameter portion 152 of the horn 130.

The controller 160 is generally located remote from the hand piece 112 and can be part of an electronic control console. The controller 160 is coupled to the hand piece 112 at the piezoelectric element assembly 134 via an electric cable or connector 168 or may be coupled via other communication means. The electronic control console is further coupled to the hand piece 112 via flexible tubing in order to provide irrigation and aspiration.

The controller 160 includes a processor 162, a memory 164, and controller circuitry 166. The processor 162 may be any type of general purpose processor or could be a processor specifically designed for the hand piece 112, such as an application-specific integrated circuit (“ASIC”). The processor 162 may be the same processor that operates the entire hand piece 112 or may be a separate processor.

The memory 164 can be any type of storage device or non-transitory computer-readable medium, such as random-access memory (“RAM”) or read-only memory (“ROM”). The memory 164 stores instructions executed by the processor 162, including instructions to provide multiple modes of oscillation simultaneously (i.e., at the same time) via the piezoelectric element assembly 134, and other functionality disclosed herein. The controller circuitry 166 also provides functionality, in addition to the functionality of the processor 162, for providing multiple modes of oscillation simultaneously via the piezoelectric element assembly 134. In example embodiments, functionality disclosed herein can be provided by the processor 162 and the memory 164 (i.e., software based), by the controller circuitry 166 (i.e., hardware based), or by a combination thereof.

The control of the modality of the ultrasonic power and the ultrasonic motion, for a hand piece such as the hand piece 112 can be implemented by a number of different methods. One method involves a control loop which servos the frequency of the drive voltage by using the electrical impedance of the piezoelectric drive transducers as feedback. In such a method, the impedance feedback of the piezo-electric transducers is computed as the ratio of the root mean square (“RMS”) value of the transducer drive voltage to the RMS value of drive current.

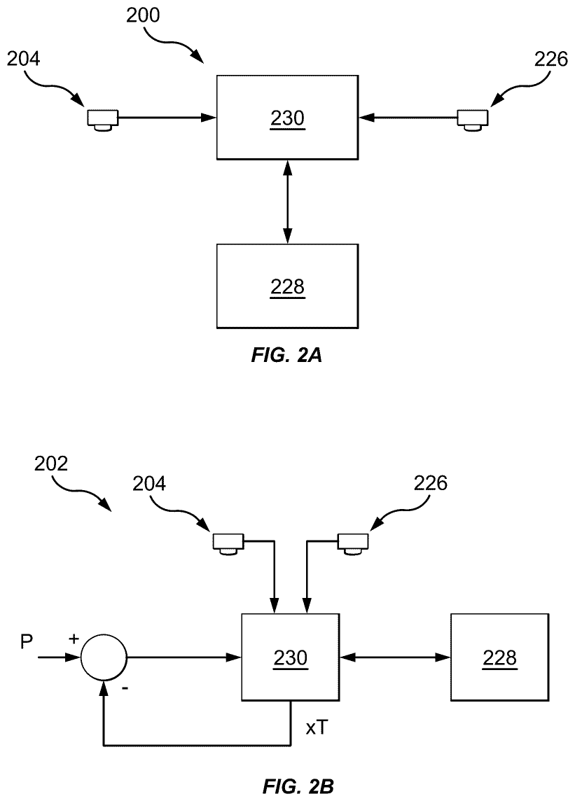

In certain embodiments, the controller 160 may be implemented as part of a control system for managing power supplied to the hand piece 112, such as controller 230 described below. Example of control systems are described in further detail with reference to FIGS. 2A-2B.

FIG. 2A is a block diagram of one embodiment of a control system 200 of the phacoemulsification system 100 (FIG. 1A), according to certain embodiments. The control system 200 includes a controller 230 coupled to a power source 228, such as an ultrasonic power source, an irrigation pressure sensor 204, and a temperature sensor 226 as described with reference to FIGS. 1A-1C. In this manner, the controller 230 is coupled to the irrigation pressure sensor 204, the temperature sensor 226, and the power source 228. Although not shown, the controller 230 may be coupled to the hand piece 112 (FIGS. 1A-1C), which may be, for example, a phacoemulsification hand piece.

In certain embodiments, the controller 230 receives irrigation pressure information from the irrigation pressure sensor 204, irrigation fluid temperature information from the temperature sensor 226, and ultrasonic power modality information from the power source 228. The controller 230 also interfaces with the power source 228 and controls its operation-thereby controlling the power provided to the hand piece 112 (FIGS. 1A-1C). In other words, the controller 230 is configured to generate a control signal provided to the power source 228 to control an ultrasonic power modality.

The ultrasonic power modality may be selected from two-dimensional (2D) power modalities, such as a longitudinal power modality, a torsional power modality, etc., and three-dimensional (3D) power modalities, such as a combination of the longitudinal power modality and the torsional power modality, etc. When the hand piece 112 operates using the longitudinal power modality, the tip of the hand piece 112 may move longitudinally (such as vertically up and down). When the hand piece 112 operates using the torsional power modality, the tip of the hand piece 112 may move rotationally (such as clockwise and counter-clockwise). When the hand piece 112 operates using the 3D power modality, the tip of the hand piece 112 may simultaneously move vertically and rotationally (such as vertically up/down and clockwise/counter-clockwise). Each power modality may increase a thermal index value differently due to different thermal behavior as a result of different shapes and/or movements of the tip of the hand piece 112.

Unwanted heating can occur at the incision site when too much power is applied to the hand piece 112 with too low of an irrigation flow rate. Since the irrigation fluid carries heat away, when the flow rate is decreased (such as when an occlusion occurs), heating can occur. Generally, the amount of heat generated is a function of the amount of power applied to the hand piece 112 and the irrigation flow rate. Friction between the irrigation sleeve and the phacoemulsification tip is the primary source of heat. When the tip rubs against the sleeve, it produces heat. The amount of power applied to the hand piece 112 is linearly related to the tip stroke—or the distance the tip travels. The more power applied, the more the tip travels (and the more the tip rubs against the sleeve).

In certain embodiments, a thermal index value (T̅(t)), which is provided in degrees Celsius (° C.), is estimated based on a double exponential Green (impulse response) function with six pre-optimized coefficients, the irrigation flow rate (F(t)), which is provided in cubic centimeters per minute (cc/min), and the ultrasonic power modality (P(t)), which is provided as a percentage (%) which may be extracted from a surgical console. That is, the thermal index value (T̅(t)) is given by Equation 1:

$$\overline{T} ( t ) = {\overline{T}}_{b s s} + {\int }_{0}^{t} ( {C}_{{p}_{1}} {e}^{- {\int }_{τ}^{t} [ {C}_{{a}_{1}} + {C}_{{f}_{1}} F ( η ) ] d η} + {C}_{{p}_{2}} {e}^{- {\int }_{τ}^{t} [ {C}_{{a}_{2}} + {C}_{{f}_{2}} F ( η ) ] d η} ) P ( τ ) d τ \qquad ( Eq . 1 )$$

where Cₚ_{₁} represents a first thermal rise coefficient; Cₚ_{₂} represents a second thermal rise coefficient; Cₐ_{₁} represents a first air decay coefficient; Cₐ_{₂} represents a second air decay coefficient; C_{f}_{₁} represents a first flow decay coefficient; C_{f}_{₂} represents a second flow decay coefficient; F represents an irrigation flow rate determined from a measured irrigation pressure; P represents a provided ultrasonic power; t and y represent dummy integration variables; and T̅_{bss} represents a temperature of the irrigation fluid, which may be supplied by a temperature sensor (e.g., temperature sensor 126 (FIG. 1B) or 226 (FIG. 2A)).

In other words, the thermal rise coefficients control thermal index value changes due to a change in a provided ultrasonic power modality, the air decay coefficients control the thermal index response to ambient conditions, and the flow decay coefficients control the thermal index response to variations of the irrigation flow rate. While the 2D longitudinal power modality and the 2D torsional power modality may linearly increase a thermal index value, the 3D power modality may non-linearly increase the thermal index value.

In this manner, the controller 230 may be configured to calculate a thermal index value based on the irrigation flow rate, the temperature of the irrigation fluid, and the ultrasonic power modality. The irrigation flow rate may be determined from the irrigation pressure. For example, since the cross section area of the irrigation path is known, the irrigation flow rate through the irrigation line may be determined from the irrigation pressure measurement as received from the irrigation pressure sensor. The temperature of the irrigation fluid may be determined based on the temperature of the irrigation fluid as received from the temperature sensor. The ultrasonic power modality may be selected by a user, such as a surgeon, etc.

This calculated thermal index value provides an estimate of the actual temperature experienced at the incision site where burning is most likely to occur. Because the temperature sensor determines the temperature of the irrigation fluid, an absolute thermal index value may be calculated instead of a relative thermal index value.

Although the calculated thermal index value is described as being based on the irrigation flow rate, the temperature of the irrigation fluid, and the ultrasonic power modality, the calculated thermal index value may also be determined without the temperature of the irrigation fluid. In other words, the control system may not include a temperature sensor, and may calculate the thermal index value based on the irrigation flow rate and the ultrasonic power modality. Thus, the calculated thermal index value (T̅(t)) may be a function of the power (P) applied to the hand piece, the irrigation flow rate (F); in other words, T̅(t)=f(P,F).

Since the calculated thermal index value provides an estimate of the actual temperature, a threshold value (such as a threshold thermal index value) may be used to control the ultrasonic power provided to hand piece. For example, in response to (e.g., when) the calculated thermal index value reaching the threshold value, the ultrasonic power is decreased to reduce the likelihood of heating. The threshold value may be based on a sensitivity level representing a friction between an irrigation sleeve and a phacoemulsification tip of the ultrasonic hand piece. The sensitivity level may be input, set, adjusted, etc., by the user. Further, in certain embodiments, the thermal index (T̅(t)) may be compared with the incision temperature rise above the ambient (ΔT) to evaluate the overall accuracy of the power control process.

As seen in FIG. 2A, the controller 230 may receive an irrigation pressure measurement from the irrigation pressure sensor 204, which is used to determine an irrigation flow rate, and the temperature of the irrigation fluid from the temperature sensor 226. Since the controller 230 controls the power source 228, the controller 230 also has the value for the power level applied to the hand piece 112 (FIGS. 1A-1C). The controller 230 uses these three values (in conjunction with the coefficient of friction) to calculate a temperature that estimates the actual temperature at the incision site. In this manner, the controller 230 continuously or periodically calculates the thermal index value, T̅(t)=f(P, F, Tp). The calculated thermal index value is compared continuously or periodically to a threshold value. In response to the calculated thermal index value reaching the threshold value, the power to the hand piece is decreased. In some embodiments, the controller 230 may be configured to adjust the thermal index value based on the temperature of the irrigation fluid over time and calculate a variation of the thermal index value above a continuous change in the temperature of the irrigation fluid.

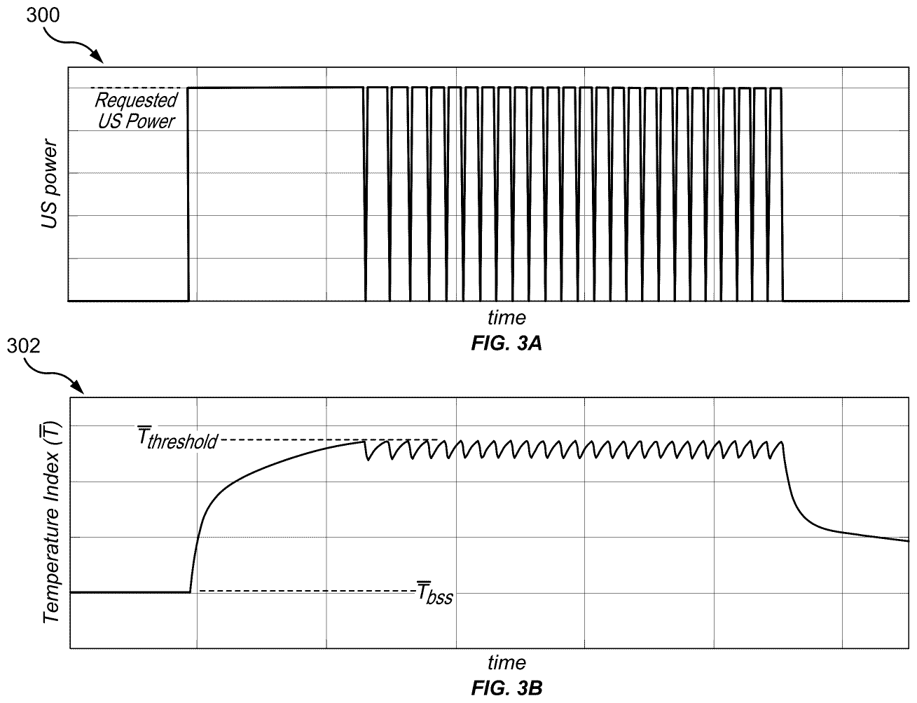

In certain embodiments, the calculated thermal index value is used as an input to control, adjust, etc., the amount of power provided to the hand piece. In this manner, the actual power applied to the hand piece tracks the inverse of the calculated thermal index value when the calculated thermal index value reaches the threshold value. In other words, the power level indicated by the control signal provided to the power source tracks an inverse of a segment of the calculated thermal index value that reaches the threshold thermal index value. This is described in more detail with reference to FIGS. 3A-3B.

As described, the threshold value may be input, set, adjusted, etc., by the user, or it can be preset. A range of threshold values may be chosen—each of which may provide a level of protection against unwanted corneal burns. For example, the highest threshold value in the range can be set at a value that provides a small difference (e.g., 1° F.) between the temperature at which the cornea burns and the threshold. A lower threshold value can be set so that the difference between the temperature at which the cornea burns and the threshold is much greater (10° F. or so).

Regardless of the threshold chosen, the ultrasonic power provided to the hand piece may be adjusted in response to the calculated thermal index value reaching the threshold value, i.e., when the calculated thermal index value is equal to or greater than the threshold value. When the calculated thermal index value is less than the threshold, the ultrasonic power provided to the hand piece is not adjusted and the ultrasonic power is governed by the selected ultrasonic power modality. Execution of this process by a control system is described in further detail with reference to FIG. 2B.

FIG. 2B is a block diagram of another embodiment of a control system 202 of the phacoemulsification system 100 (FIG. 1A), according to certain embodiments. The control system 202 shown FIG. 2B more clearly illustrates the process in operation.

The controller 230 calculates the calculated thermal index value based on data received from the irrigation pressure sensor 204, data received from the temperature sensor 226, and the power from the power source 228. In FIG. 2B, the controller 230 may provide a proportional-integral (PI) control process, a proportional-integral-derivative (PID) control process, etc. The inverse of the scaled calculated thermal index value may be subtracted from the power to reduce the power applied to the hand piece. In this manner, the controller 230 controls the output of the power source 228 by adjusting the control signal provided to the power source 228.

The adjusted control signal thereby indicates a decrease in the amount of power output by the power source 228 by an amount that is inversely proportional to the calculated thermal index value (or by an amount that is inversely proportional to the thermal index value in excess of the threshold)—designated by xT—where x can be a scalar or a function. In this manner, when the calculated thermal index value reaches the threshold value, the power supplied to the hand piece is decreased in proportion to an amount that is in excess of the threshold value. When the calculated thermal index value is less than the threshold value, normal operation resumes.

The control process may execute during an ophthalmic surgery. Generally, during surgery, the doctor manually controls the application of ultrasonic power to the hand piece, such as by using a foot pedal. When the calculated thermal index value reaches the threshold value, the control process may override manual power control, and, when the calculated thermal index value is less than the threshold value, manual power control may be resumed.

The control process advantageously allows surgeons to easily focus on the ophthalmic surgery by automatically reducing the ultrasonic power supplied to the hand piece if the threshold is reached, which reduces the likelihood of burns at the incision site. As a result of this non-invasive control process, surgeons may continue procedures and maintain thermal safety at the same time. In addition, because the control process may be adjusted according to different levels of sensitivities, the control process provides flexibility. Further, the control process is an additional safety feature which may be beneficial for less experienced surgeons.

FIGS. 3A-3B are graphs depicting an exemplary operation of thermal management by the control systems 200 (FIG. 2A) and 202 (FIG. 2B), according to certain embodiments. FIGS. 3A-3B are described together herein for clarity purposes. FIG. 3A is a graph 300 depicting an amount of ultrasonic power (y-axis) over time (x-axis) in response to a calculated thermal index value. FIG. 3B is a graph 302 depicting the calculated thermal index value (or temperature index (T̅)) (y-axis) over time (x-axis) during operation of a hand piece (e.g., hand piece 112).

In certain embodiments, the amount of ultrasonic power shown in FIG. 3A represents a power level that is specified by a control signal provided to the power source 228 by the controller 230. The power level specified by the control signal may be based on the ultrasonic power modality. In certain embodiments, the calculated thermal index value shown in FIG. 3B represents a calculated temperature at a tip of the hand piece 112, which may be calculated by the controller 230.

Prior to operation of the hand piece 112, the calculated thermal index value may be equal to a temperature of the irrigation fluid (T̅_{bss}). When the calculated thermal index value is below a threshold value (T̅_{threshold}), a surgeon can apply an ultrasonic power modality to the hand piece. As an example, the ultrasonic power modality may be a 2D power modality or a 3D power modality and may be regulated by the foot pedal. As depicted, the surgeon is applying 100% power to the hand piece, which is adjusted from 0% to 100% by manipulating the foot pedal connected to the controller. Additionally, the power may be applied to the hand piece via a power application technique while the foot pedal is depressed. For example, the power application technique may be a continuous mode, a burst mode, or a pulse mode.

When the calculated thermal index value reaches the threshold value, the control process overrides the surgeon's control of power to prevent the calculated thermal index value from further exceeding the threshold value. When the power is decreased, the calculated thermal index value will tend to decrease as well. When the calculated thermal index value is less than the threshold value, the surgeon resumes manual power control—in this case, power applied returns to 100%.

In continuous mode, the degree to which the foot pedal is depressed, or the position of the foot pedal, determines the amount of power or power level applied. The control process decreases the power in proportion to the temperature rise over the threshold value. In other words, an incremental temperature increase over the threshold value results in a proportional decrease in the amount of power applied to the hand piece. The decrease in power can be smooth, such that a smooth decrease in power still results in power being applied smoothly to the tip of the hand piece.

In pulse mode, a series of fixed width power pulses is applied to the hand piece. The surgeon controls the amplitude, or power level, of the pulses with the foot pedal. In this manner, the position of the foot pedal determines the power level of the pulses. The control process may decrease the power of any given pulse non-linearly, such that the process operates on an individual pulse, or a series of pulses as the case may be.

In some embodiments, an incremental temperature increase over the threshold value results in a proportional decrease in the amount of power applied to the hand piece operating in the pulse mode. The decrease in power can be incremental, such that an incremental decrease in power still results in power being applied to the cutting tip of the hand piece. The control process may operate to decrease the power of the next pulse while maintaining a constant pulse level, such that the control process operates on the next pulse and serves to limit the power level of that next pulse to a constant power level.

In burst mode, a series of pulses is applied to the hand piece. The surgeon controls the off time between pulses with the foot pedal. In this manner, the position of the foot pedal determines the off time between the pulses. The control process decreases the power in proportion to the temperature rise over the threshold value. In other words, an incremental temperature increase over the threshold value results in a proportional decrease in the amount of power applied to the hand piece. In this manner, a smooth decrease in power still results in power being applied smoothly to the cutting tip of the hand piece. The control process decreases the power of any given pulse non-linearly, such that the process operates on an individual pulse or a series of pulses.

In some embodiments, an incremental temperature increase over the threshold value results in a proportional decrease in the amount of power applied to the hand piece operating in the burst mode. The decrease in power can be incremental, such that an incremental decrease in power still results in power being applied to the cutting tip of the hand piece. The control process decreases the power of the next pulse while maintaining a constant pulse level, such that the control process operates on the next pulse and serves to limit the power level of that next pulse to a constant power level.

Several variations of the control process may also be implemented. In one embodiment, the power is decreased in proportion to a scalar factor of the temperature increase. In another embodiment, the power is decreased in proportion to a function of the temperature increase. In another embodiment, a minimum power level can be set, and, in this case, power will not fall below the minimum power level resulting in a continuous but lower application of power to the hand piece. In yet another embodiment, the rate at which power is decreased may be changed, and, in this case, the power decrease is smooth. A smooth decrease in power results in more effective cutting, as power is applied continuously (i.e., not turned off) which may feel better in the surgeon's hand.

From the above, it may be appreciated that the present disclosure provides a thermal management process for phacoemulsification surgery. The present disclosure provides a control system that calculates a thermal index value, compares the calculated thermal index value to a threshold value, and reduces power supplied to the hand piece when the calculated thermal index value reaches the threshold value. The present disclosure is illustrated herein by example, and various modifications may be made by a person of ordinary skill in the art.

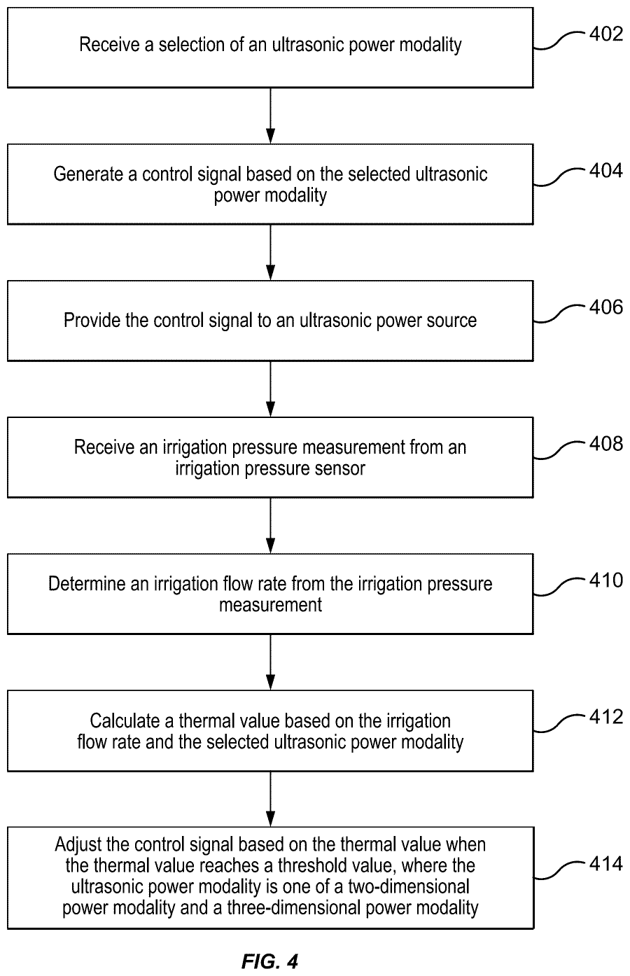

FIG. 4 illustrates a flow diagram of a technique for controlling power supplied to an ultrasonic hand piece, according to certain embodiments.

At block 402, a selection of an ultrasonic power modality is received. The selection of the ultrasonic power modality may be received via an input of a user and transmitted to the controller 230. That is, the user may input the selected ultrasonic power modality, which is then transmitted as digital data to the controller 230. The controller 230 then receives the digital data with the selected ultrasonic power modality. The ultrasonic power modality is one of a two-dimensional power modality and a three-dimensional power modality.

At block 404, a control signal is generated based on the selected ultrasonic power modality, as described above. The control signal may be generated by the controller 230 after the controller has received the selected ultrasonic power modality at block 402.

At block 406, the control signal is provided to an ultrasonic power source. The control signal may be provided by the controller 230 to the power source 228. For example, the controller 230 may transmit the control signal as digital data or as an analog signal to the power source 228.

At block 408, an irrigation pressure measurement is received from an irrigation pressure sensor. The irrigation pressure may be measured by the irrigation pressure sensor 204 and transmitted by the irrigation pressure sensor 204 to the controller 230. The irrigation pressure sensor 204 may record the irrigation pressure measurement and convert it into digital data, which is then transmitted as measured digital data (or as an analog signal) to the controller 230. The controller 230 receives the measured digital data and may then analyze the irrigation pressure measurement.

At block 410, an irrigation flow rate is determined from the irrigation pressure measurement. The irrigation flow rate may be determined by the controller 230 using the irrigation pressure measurement received from the irrigation pressure sensor 204 at block 408.

At block 412, a thermal index value is calculated based on the irrigation flow rate and the selected ultrasonic power modality, as described above. The thermal index value may be calculated by the controller 230. That is, the controller 230 may calculate the thermal index value based on the irrigation flow rate determined from the measured pressure data provided by the irrigation pressure sensor 204, and the selected ultrasonic power modality inputted by a user.

At block 414, when the thermal index value reaches a threshold value, the control signal is adjusted based on the thermal index value. The controller 230 may adjust the control signal. That is, the controller 230 may compare the calculated thermal index value to the threshold value, and if the calculated thermal index value is greater than or equal to the threshold value, then the controller 230 adjusts the control signal.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

It will be readily understood that the components of the embodiments as generally described herein and illustrated in the Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

What is claimed is:

1. A control system for an ultrasonic hand piece, the control system comprising:
an irrigation pressure sensor configured to measure a pressure of an irrigation fluid;
an ultrasonic power source configured to provide power to an ultrasonic transducer; and
a controller, coupled to the irrigation pressure sensor and the ultrasonic power source, configured to:
receive a selection of an ultrasonic power modality,
generate a control signal based on the selected ultrasonic power modality,
provide the control signal to the ultrasonic power source,
determine an irrigation flow rate from a measured irrigation pressure provided by the irrigation pressure sensor,
calculate a thermal index value based on the irrigation flow rate and the selected ultrasonic power modality, and
in response to the thermal index value reaching a threshold value, adjust the control signal based on the thermal index value,
wherein the ultrasonic power modality is one of a two-dimensional power modality and a three-dimensional power modality.

2. The control system of claim 1, further comprising:
a temperature sensor configured to measure a temperature of the irrigation fluid,
wherein said calculate the thermal index value is further based on the measured temperature.

3. The control system of claim 2, wherein the controller is further configured to:
adjust the thermal index value based on the temperature of the irrigation fluid over time; and
calculate a variation of the thermal index value above a continuous change in the temperature of the irrigation fluid.

4. The control system of claim 1, wherein the two-dimensional power modality is one of a longitudinal power modality and a torsional power modality.

5. The control system of claim 4, wherein the three-dimensional power modality is a combination of the longitudinal power modality and the torsional power modality.

6. The control system of claim 5, wherein said calculate the thermal index value includes non-linearly increasing the thermal index value when the selected ultrasonic power modality is the three-dimensional power modality.

7. The control system of claim 1, wherein:
the control signal includes a power level; and
said adjust the control signal includes adjust the power level in proportion to the thermal index value.

8. The control system of claim 7, wherein said adjust the power level in proportion to the thermal index value includes tracking an inverse of a segment of the thermal index value that reaches the threshold value.

9. The control system of claim 7, wherein:
the controller is further configured to adjust the power level based on the ultrasonic power modality.

10. The control system of claim 9, wherein the controller is further configured to:
when the thermal index value reaches the threshold value, override a manual power control of the ultrasonic power source; and
when the thermal index value is less than the threshold value, resume the manual power control of the ultrasonic power source.

11. The control system of claim 1, wherein the threshold value is based on a sensitivity level representing a friction between an irrigation sleeve and a phacoemulsification tip of the ultrasonic hand piece.

12. A method for controlling power supplied to an ultrasonic hand piece, the method comprising:
receiving a selection of an ultrasonic power modality;
generating a control signal based on the selected ultrasonic power modality;
providing the control signal to an ultrasonic power source;
receiving an irrigation pressure measurement from an irrigation pressure sensor;
determining an irrigation flow rate from the irrigation pressure measurement provided by the irrigation pressure sensor;
calculating a thermal index value based on the irrigation flow rate and the selected ultrasonic power modality; and
in response to the thermal index value reaching a threshold value, adjusting the control signal based on the thermal index value,
wherein the ultrasonic power modality is one of a two-dimensional power modality and a three-dimensional power modality.

13. The method of claim 12, further comprising:
receiving a temperature measurement of an irrigation fluid from a temperature sensor,
wherein said calculating the thermal index value is further based on the measured temperature.

14. The method of claim 12, wherein
the two-dimensional power modality is one of a longitudinal power modality and a torsional power modality; and
the three-dimensional power modality is a combination of the longitudinal power modality and the torsional power modality.

15. The method of claim 12, wherein said calculating the thermal index value includes non-linearly increasing the thermal index value when the selected ultrasonic power modality is the three-dimensional power modality.
